(19) United States
(12) Patent Application Publication
(10) Pub. No.: US 2003/0077298 A1
(43) Pub. Date: Apr. 24, 2003

(54) Activators and ligands of PPAR-beta/delta for the treatment of skin conditions

(75) Inventors: Peter M. Elias, Mill Valley, CA (US); Kenneth R. Feingold, San Rafael, CA (US); Matthias Schmuth, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, a California Corporation, Oakland, CA

(21) Appl. No.: 10/121,954

(22) Filed: Apr. 12, 2002

Related U.S. Application Data
(60) Provisional application No. 60/283,825, filed on Apr. 13, 2001.
(60) Provisional application No. 60/283,851, filed on Apr. 13, 2001.

(51) Int. Cl. A61K 31/426; A61K 31/421; A61K 9/00

(52) U.S. Cl. 424/400; 514/365; 514/374

(57) ABSTRACT

Disorders of the skin and mucous membrane that have a disrupted or dysfunctional epidermal barrier, disturbed differentiation, or inflammation are treated or prevented by topical application of compounds that are activators of peroxisome proliferator-activated receptor-delta.

### CROSS-REFERENCES TO RELATED APPLICATIONS

This application claims priority to U.S. Ser. No. 60/283,825, filed Apr. 13, 2001, and to U.S. Ser. No. 60/283,851 filed Apr. 13, 2001 each of which is herein incorporated by reference in its entirety.

### STATEMENT AS TO RIGHTS TO INVENTIONS MADE UNDER FEDERALLY SPONSORED RESEARCH AND DEVELOPMENT

This invention was made at least in part with assistance from the United States Federal Government, under Grant No. AR39448, and HD29706 of the National Institutes of Health. As a result, the government has certain rights in this invention.

### FIELD OF THE INVENTION

This invention resides in the technical field of therapeutic agents for the treatment of subjects suffering from skin or mucous membrane diseases or disorders that involve epidermal differentiation and proliferation, disruptions of epidermal barrier function, or cutaneous inflammation.

### BACKGROUND OF THE INVENTION

Peroxisome proliferator activated receptors (PPARs) function as ligand-activated transcription factors. These transcription factors regulate genes important in metabolism and differentiation (Kersten et al., 2000, Nature 405:421).

Peroxisome proliferator-activated receptors (PPAR) are described in a review article by Schoonjans, K., “Role of the peroxisome proliferator-activated receptor (PPAR) in mediating the effects of fibrates and fatty acids on gene expression,” J. Lipid Res. 37:907-925 (1996). Three subtypes of PPAR have been identified and are designated α, β (or δ), and γ, respectively. The α subtype has been cloned from Xenopus, humans, mouse and rat; the β (or δ) subtype from Xenopus, humans and mouse; and the γ subtype from Xenopus, humans and hamster. The PPARs have a modular structure consisting of six functional domains. The one domain that serves as the DNA-binding domain contains about 66 amino acids and is stabilized by two zinc atoms, each binding to four invariant cysteine residues. The ligand binding domain is a large multifunctional domain that functions in dimerization and nuclear localization in addition to ligand binding.

The peroxisome proliferator-activated receptors (PPARs) are members of the class II subset of the nuclear hormone receptor superfamily of transcription factors. The peroxisome proliferator-activated receptors function only when having formed heterodimers with the retinoid X receptor (RXR). In addition to the PPARs, other members of the class II subset of the nuclear hormone receptor superfamily that form heterodimers with the retinoid X receptor (RXR) include the vitamin D receptor (VDR), the all-trans-retinoic acid receptors (RAR α,β,δ) and the liver X receptor.

PPARs are activated by fatty acids and other lipid metabolites. Thus, they function as “liposensors” (Mangelsdorf and Evans, 1995, Cell 83:841; Chawla et al., 2001, Science 294:1866). PPAR-alpha is expressed in liver, heart, kidney, muscle and brown fat (Schoonjans et al., 1996, Biochim Biophys Acta 1302:93; Gonzalez, 1997, Adv Exp Med Biol 422:109), as well as in the epidermis and its appendages (Deplewski and Rosenfield, 2000, Endocr Rev 21:363; Billoni et al., 2000, Acta Derm Venereol 80:329). PPAR-gamma is predominantly expressed in adipocytes, colon, spleen, and adrenal tissue, where it mediates free fatty acid uptake, lipogenisis and differentiation (Schoonjans et al., 1996, Biochim Biophys Acta; Kersten et al., 2000, Nature 405:421; Rosen and Spiegelman, 2001, J Biol Chem 276:37731). PPAR-beta/delta is expressed ubiquitously (Kliewer et al., 1994, PNAS USA 91:7355-9) and has recently been shown to increase serum high density lipoprotein cholesterol levels while lowering serum triglyceride and insulin levels when administered to obese rhesus monkeys (Oliver et al., 2001, PNAS USA 98:5306).

Some members of the class II subset of the nuclear hormone receptor superfamily are known to function in modulation of cutaneous homeostasis. For example thyroid receptor, retinoic acid receptor, and vitamin D receptor are well known regulators of epidermal growth and development (Pillai and Bikle, 1991, Adv Lipid Res 24:321; Blumenberg et al. 1992, J Invest Dermatol 98:42S; Torma et al., 1993, Acta Derm Venereol 73:102; Fisher and Voorhees, 1996, FASEB J 10:1002). Activators of retinoic acid receptor, and vitamin D receptor are currently employed to treat a variety of skin diseases (Frisch, 1992, J Am Acad Dermatol 27:S8; Kragballe, 1992, J Cell Biochem 49:46; Orfanos et al., 1997, Drugs 53:358; Saurat, 1999, J Am Acad Dermatol 41:S2). And, activators of the peroxisome proliferator-activated receptor-alpha (PPARα) have been disclosed as therapeutic agents in the treatment of epidermal disorders that involve disruptions of the epidermal barrier function, and in the treatment of disorders of epidermal differentiation and proliferation. (See e.g. U.S. Pat. No. 6,060,515, “Treatment of Skin conditions by use of PPARα Activators,” issued May 9, 2000, on an application filed Jun. 16, 1999, Elias et al., inventors).

Aside from its role in lipid metabolism as noted above, the function of PPAR-beta/delta, is unknown. PPAR-beta/delta is abundantly expressed in keratinocytes (Rivier et al., 1998, J Invest Dermatol 111:1116; Braissant and Wahli, 1998, Endocrinology 139:2748; Westergaard et al., 2001, J Invest Dermatol 116:702), but neither its effects on keratinocyte growth and differentiation, nor its impact on inflammatory processes in these cells are known.

### BRIEF SUMMARY OF THE INVENTION

It has now been discovered that epidermal inflammation, restoration of barrier function and alterations of epidermal differentiation that arise from skin diseases or conditions can be controlled, eliminated, or prevented by the topical administration of activators of the peroxisome proliferator-activated receptor-delta (PPARδ) that are not also activators of peroxisome proliferator-activated receptor α (PPAR α).

The peroxisome proliferator-activated receptors (PPARs) are members of the nuclear hormone receptor superfamily that form heterodimers with the retinoid X receptor (RXR). These emerging transcription factors are believed to act as “liposensors” that mediate lipid metabolism and cellular differentiation. Since little is known about the function of PPAR-beta/delta, the discovery that the activators of PPAR-beta/delta are effective in controlling epidermal differentiation and inflammation is unexpected.

### BRIEF DESCRIPTION OF THE DRAWINGS

FIG. 1 illustrates the changes in the levels of involucrin, transglutaminase I, and PPAR-beta/delta mRNA that occur when cultured human human keratinocytes are grown in the presence of GW1514. Changes in the levels of involucrin protein that occur when cultured human human keratinocytes are grown in the presence of GW1514, are also shown.

FIG. 2 illustrates filaggrin and loricrin staining of vehicle treated or GW1514 treated normal epidermis.

FIG. 3 is a bar graph illustrating the number of PCNA positive nuclei in GW1514 treated normal epidermis

FIG. 4 illustrates fillagrin and loricrin staining of epidermis from wild type and PPAR-alpha knockout mice that has been topically treated with GW1514 or vehicle alone.

FIG. 5 illustrates percent barrier recovery as a function of time of epidermis topically treated with GW1514 or vehilcle alone following epidermal barrier disruption by repeated tape-stripping, repeated solvent treatment, or repeated treatment with detergent.

FIG. 6 illustrates filaggrin and loricrin staining and PCNA staining of proliferating nuclei in skin experiencing experimentally induced hyperplasia which has been treated by topical application of GW1514 or vehicle alone.

FIG. 7 is a graphical representation of the number of PCNA positive nuclei in in skin experiencing experimentally induced hyperplasia which has been treated by topical application of GW1514 or vehicle alone.

FIG. 8 is a bar graph illustrating induction of PPAR-beta/delta mRNA in cultured human keratinocytes grown in the presence of the inflammatory stimuli indicated. Results are shown as percent of vehicle only control.

FIG. 9 illustrates ear thickness as percent vehicle control.

FIG. 10 is a bar graph illustrating the relative levels of PPAR alpha, gamma, and delta in skin scrapings from psoriatic lesions and normal skin of psoriasis patients.

### DETAILED DESCRIPTION

The peroxisome proliferator-activated receptor-delta (PPAR δ) belongs to the nuclear hormone receptor superfamily of transcriptional regulators. This group of approximately 150 proteins bind to cis-acting elements of the promoters of their target genes to modulate gene expression in response to hormone activators or ligands. The superfamily is divided into two major subgroups based on cellular localization of the unliganded form. The class I receptors are localized in the cytoplasm, and upon binding of their ligand are transported to the nucleus. The class II receptor subgroup, of which PPARs are members, are located in the nucleus. Upon binding of a ligand in the nucleus, the conformation of the receptor changes such that activation of target genes is possible.

Amongst the class II nuclear receptor subgroup are those receptors that bind their target genes as heterodimers. Prominent among the heterodimeric receptors are nuclear receptors that require heterodimerization with the retinoid X receptor, as disclosed by Yu, V. C., et al., “RXRβ: a coregulator that enhances binding of retinoic acid, thyroid hormone, and vitamin D receptors to their cognate response elements,” Cell 67:1251-1266 (1991). Members of this group include the vitamin D receptor, the thyroid hormone receptor (T₃R), the retinoic acid receptor (RAR), the farnesoid X-activated receptor (FXR), LXRα/β and the peroxisome proliferator-activated receptors (PPAR).

Peroxisome proliferator-activated receptors (PPAR) are described in a review article by Schoonjans, K., “Role of the peroxisome proliferator-activated receptor (PPAR) in mediating the effects of fibrates and fatty acids on gene expression,” J. Lipid Res. 37:907-925 (1996). Three subtypes of PPAR have been identified and are designated α, β (or δ), and γ, respectively. The α subtype has been cloned from Xenopus, humans, mouse and rat; the β (or δ) subtype from Xenopus, humans and mouse; and the γ subtype from Xenopus, humans and hamster.

The PPARs have a modular structure consisting of six functional domains. The DNA-binding domain contains about 66 amino acids and is stabilized by two zinc atoms, each binding to four invariant cysteine residues. The DNA binding domain is highly conserved among all members of the nuclear receptor superfamily. The ligand binding domain of the PPARs is also important in dimerization and nuclear localization. The ligand binding domain is less conserved than the DNA binding domain. The ligand binding domain varies within the group of class II nuclear receptors as well as within the PPAR family itself. The lack of conservation of the ligand binding domain within the PPAR family probably reflects the fact that the PPARs bind similar but not identical ligands.

Upon activation by fatty acids and drugs that affect lipid metabolism, PPARs control expression of genes implicated in intra- and extracellular lipid metabolism, most notably those involved in peroxsomal β-oxidation. Thus, fatty acids have been proposed to be the endogenous PPAR activators. Detailed knowledge of the ligand activators of some receptors are known, but for others, termed “orphan receptors,” the activators are unknown. For PPAR-gamma the natural activators and ligands include acrachadonic acid metabolites of the prostaglandin J2 group. For PPAR-alpha some natural activators are fatty acids other than short chain (<C10) fatty acids. Other activators of PPAR-alpha are also known (see e.g. U.S. Pat. No. 6,060,515 to Elias et al.). For PPAR-beta/delta however, very little is known about the specific nature of any endogenous or exogenous activators or ligands.

Many of the available PPAR-agonists display some degree of cross-reactivity between members of the PPAR family. For example, WY 14643 activates both PPAR-alpha and PPAR-beta/delta. Similarly, tetradecylthioacetic acid activates all three receptor subtypes. It has now been discovered however, that there are two preferred activators of PPAR-beta/delta that are not also activators of PPAR-alpha. These are: (2-methyl-4-(((4-methyl-2-(4-trifluoromethylphenyl)-1,3-oxazol-5-yl)methyl)sulfanyl)phenoxy)acetic acid, also known as GW 1514 and available from GlaxoSmithKline (United Kingdom, USA), whose molecular formula is

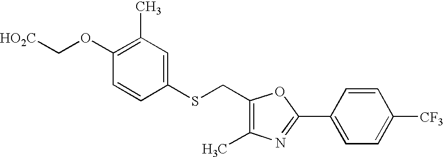

and (2-methyl-4-(((4-methyl-2-(4-trifluoromethylphenyl)-1,3-thiazol-5-yl)methyl)sulfanyl)phenoxy)acetic acid, also known as GW 501516 and available from GlaxoSmithKline (United Kingdom, USA), whose molecular formula is

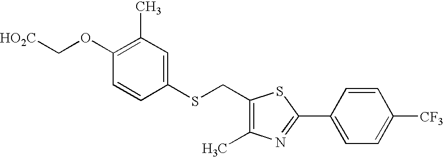

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

Interestingly, these activators have been found to stimulate keratinocyte differentiation in vitro and in vivo, without having any effect on proliferation. Furthermore, these activators are shown herein to promote the recovery of epidermal barrier function after perturbation by a variety of means. These activators are also shown herein to effectively reduce the symtoms of cutaneous inflammation when they are applied topically to affected epidermis. These findings are quite surprising given the general lack of knowledge regarding the activators and ligands of PPAR-beta/delta.

In some of its embodiments, this invention resides in the administration of these compounds for the treatment of conditions that involve or give rise to a disrupted or dysfunctional barrier function. Examples of these conditions are:

fluid and electrolyte abnormalities, hypothermia, and infection through the skin in premature infants less than 33 weeks of gestational age

inflammation to mucous membranes, such as cheilitis, chapped lips, nasal irritation and vulvovaginitis

eczematous dermatides, such as atopic and sebhorreic dermatitis, allergic or irritant contact dermatitis, eczema craquelée, photopallergic dermatitis, phytotoxic dermatitis, phytophotodermatitis, radiation dermatitis, and stasis dermatitis

ulcers and erosions resulting from trauma, burns, bullous disorders, or ischemia of the skin or mucous membranes

ichthyoses

epidermolysis bullosae

psoriasis

hypertrophic scars and keloids

cutaneous changes of intrisic aging and photoaging

frictional blistering caused by mechanical shearing of the skin

cutaneous atrophy resulting from the topical use of corticosteroids

In other embodiments, this invention resides in the administration of these compounds for the treatment of skin disorders that involve abnormal differentiation, or which would benefit from enhanced or improved epidermal differentiation. Examples of such conditions are psoriasis, atopic dermatitis, irritant contact dermatitis, various types of ichthyosis with or without an associated barrier abnormality, skin aging and benign neoplasms such as warts, condylomata, and sebhorrheic keratoses.

This invention is also useful in the treatment of cutaneous inflammatory conditions. Of particular interest in this regard is the treatment of conditions such as:

vasculitis

cutaneous lupus

panniculitis

UV-erythema, sunburn

alopecia areata/scarring alopecias

lichen planus

cheilitis

conjunctivitis

keratitis

balanitis

intertrigo

vaginitis

psoriasis

In the practice of this invention, the PPAR-beta/delta activators will be administered as active ingredients in a formulation that is pharmaceutically acceptable for topical administration. These formulations may or may not contain a vehicle, although the use of a vehicle is preferred. Preferred vehicles are non-lipid vehicles, particularly a water-miscible liquid or mixture of liquids. Examples are methanol, ethanol, isopropanol, ethylene glycol, propylene glycol, and butylene glycol, and mixtures of two or more of these compounds.

The concentration of active ingredient in the vehicle is not critical to this invention and may vary widely while still achieving a therapeutic effect, a preventive effect, or both. In most cases, concentrations within the range of from about 1 mM to about 50 mM, preferably from about 1 mM to about 20 mM, and most preferably from about 2 mM to about 10 mM, will give the best results.

The invention is generally applicable to the treatment of the skin of terrestrial mammals, including for example humans, domestic pets, and livestock. The invention is of particular interest in treating humans for the conditions described above or for preventing these conditions from becoming manifest.

### EXAMPLES

The following examples are offered for purposes of illustration, and are not intended to limit or to define the invention. All literature citations in these examples and throughout this specification are incorporated herein by reference for all legal purposes to be served thereby.

### Example 1

This example shows that the PPAR-beta/delta activator GW1514, stimulates keratinocyte differentiation in vitro.

Increased expression of the proteins involucrin and transglutaminase I is a classic indicator of in vitro keratinocyte differentiation. Therefore, to determine if GW1514 could stimulate keratinocyte differentiation, in vitro changes in the levels involucrin and transglutaminase I mRNA, and involucrin protein expression were determined when cells were cultured in the presence of GW1514. In addition, levels of PPAR-betaldelta mRNA were compared in cells treated with GW1514 versus untreated controls.

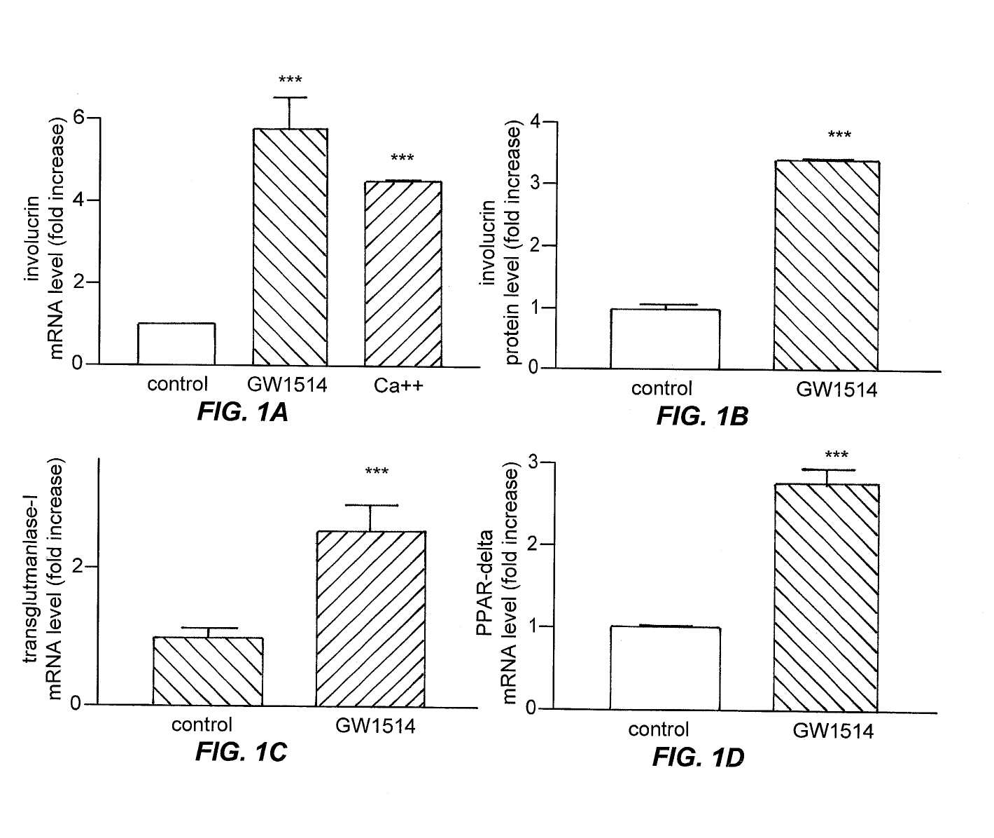

Second-passage keratinocytes isolated from newborn foreskins were cultured in serum-free keratinocyte growth medium (KGM; Clonetics, San Diego, Calif., USA). Cells were treated with the PPAR-beta/delta activator GW1514 (8 μM) or vehicle (0.05% ethanol). Poly-A mRNA was isolated and transglutaminase I, involucrin and PPAR-beta/delta mRNA levels were measured by Northern blot analysis (FIG. 1).

PolyA RNA was purified using oligo(dT) cellulose (Amersham Pharmacia). RNA was electrophoresed through 1% agarose gels containing 2.2 M formaldehyde, and RNA integrity was visualized by ethidium bromide staining. The RNA was then transferred to a nylon membrane and blots were hybridized overnight at 65° C. with the appropriate ³²P-labeled cDNA probes. Probes were ³²P-labeled by random priming. Washes were performed in 0.1% SSC and 0.1 SDS for 20 minutes at room temperature and at 65° C., respectively. Autoradiography was performed at −70° C. Blots were probed with GAPDH to control for normal loading. Bands of the correct size were quantified by densitometry using QuantityOne software (Bio-Rad Laboratories Inc., Hercules, Calif., USA).

Total cellular protein was isolated from cultured keratinocytes and involucrin protein levels were assessed by Western blot analysis. The cells were homogenized by sonication, their protein content was determined, and gels were loaded with equal amounts per sample and lane. Western Immunoblotting was performed using 7.5% SDS-PAGE, as described previously (Laemmli, 1970). Following transfer of protein to PVDF membranes, blots were incubated overnight with primary antibody at 4° C. Secondary antibody (peroxidase conjugated anti mouse; Amersham PharmaciaBiotech Inc., Piscataway, N.J.) was applied and blots were incubated at room temperature for 2 hours. Final detection was performed by chemiluminescence (ECL kit; Amersham). Involucrin protein was detected with a monoclonal mouse anti-human involucrin antibody.

Northern analysis revealed increased levels of involucrin, transglutaminase I and PPAR-beta/delta mRNA in cultured human keratinocytes incubated with GW1514. Similarly, western blots showed a 5-fold increase in involucrin protein expression (FIG. 1). Strikingly, the induction of differentiation markers by GW1514 was more pronounced than the induction achieved through the addition of high calcium concentrations to the culture medium (FIG. 1). Addition of high calcium concentrations to the culture medium is a standard, well established method for inducing keratinocyte differentiation in vitro (Yuspa et al., 1988).

These results show that PPAR-beta/delta effectively induces keratinoctye differentiation in vitro. These results suggest that PPAR-beta/delta activators could be effective in the treatment of skin conditions that are characterized by abnormal differentiation or which require the induction of keratinocyte differentiation.

### Example 2

This example shows that topical treatment of normal epidermis with the PPAR-beta/delta activator GW1514, results in increased expression of the in vivo differentiation markers filaggrin and loricrin. Altough topical treatment with GW1514 stimulates differentiation of keratinocytes, it does not stimulate their proliferation.

The skin of 6-8 week old hairless mice (Simonsen Laboratories, Gilroy, Calif.) was treated twice a day for 6 days either with vehicle (acetone) alone or with GW1514 at 4 mM concentration in the vehicle. At the conclusion of the experiment the animals were sacrificed. Mouse skin samples were fixed with 4% formaldehyde in phosphate-buffered saline (PBS) and embedded in paraffin. Sections (6 μm) were collected on microscope slides and used for immunohistochemistry. Photographs were taken with a Zeiss microscope and digital images were captured and assembled by appropriate software. Fillagrin and loricrin protein expression were detected by immunohistochemistry using antibodies purchased from BabCo (Richmond, Calif.). Proliferating keratinocytes were quantified in sections by staining with biotinylated anti-proliferating cell nuclear antigen (PCNA) antibody (CalTag Laboratories, Burlingame, Calif.).

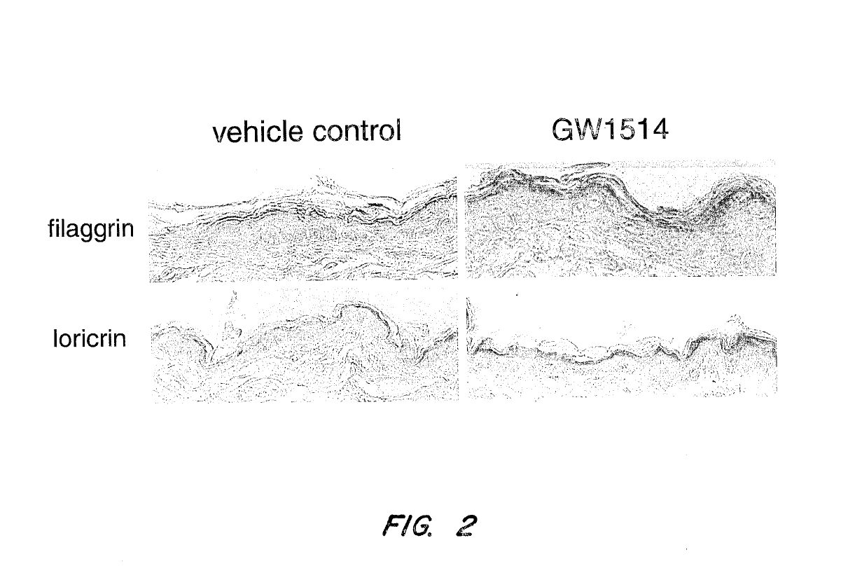

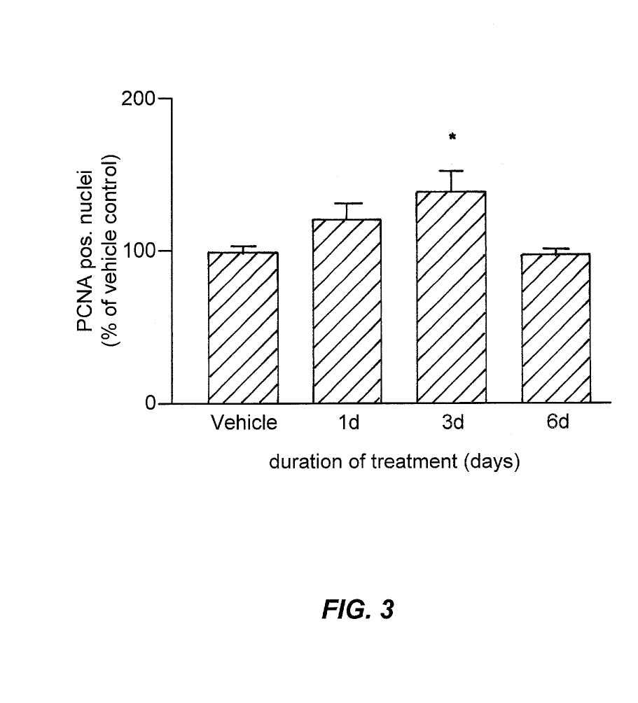

The results are shown in FIGS. 2 and 3. As can be seen in FIG. 2, mouse skin topically treated with GW1514 showed increased levels of immunohistochemical staining with filaggrin and loricrin antibodies compared to mouse skin treated with vehicle alone. Thus, topical treatment of normal epidermis with GW1514 resulted in increased expression of filaggrin and loricrin. This increased expression of in vivo differentiation markers was also apparent after only three days of treatment with GW1514.

Although the pool of proliferating cells was transiently increased at three days after treatment with GW1514, after six days treatment cell proliferation was at normal levels. Thus, topical application of the PPAR-beta/delta activator GW1514 to normal epidermis stimulate differentiation without also inducing proliferation.

These results show that topical application of GW1514 stimulates expression of epidermal differentiation markers in vivo. Thus, these results confirm the in vitro studies of Example 1. Further, these results show that on the whole, GW1514 does not induce proliferation of keratinocytes. Thus, PPAR-beta/delta activators can provide effective treatment for skin conditions such as skin aging that benefit from the induction of epidermal differentiation. PPAR-beta/delta activators can also provide effective treatment of skin conditions for conditions which involve abnormal differentiation, or which require improved differentiation without increased proliferation.

### Example 3

This example demonstrates that topical treatment of normal epidermis with the PPAR-beta/delta activator GW1514, specifically stimulates epidermal differentiation independent of any PPAR-alpha activity.

Since many of the PPAR agonists display some degree of cross-reactivity between PPAR subtypes, and since PPAR-alpha activation is known to improve barrier function (See e.g. U.S. Pat. No. 6,060,515) experiments were conducted to rule out the possibility that the activity of PPAR-alpha was being stimulated by GW1514. The epidermis of PPARα knockout (PPARα−/−) mice, as well as the epidermis of wild type(PPARα+/+) mice was treated with topical applications of GW1514. Expression of the in vivo differentiation markers loricrin and filaggrin was measured by immunohistochemical staining following treatment.

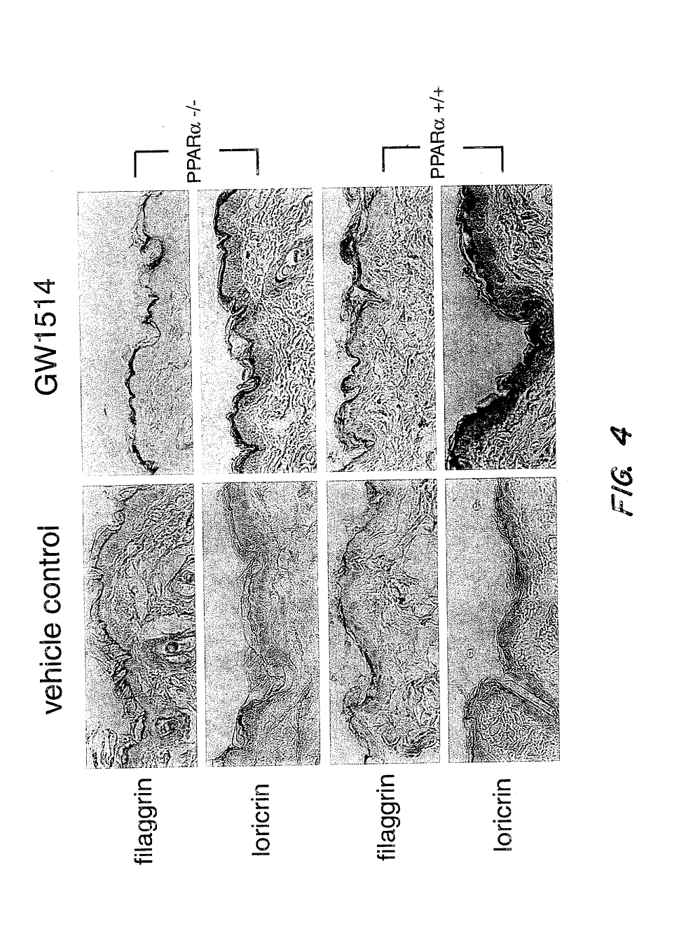

PPAR-alpha−/−mice in a mixed C57BL/6 and Sv129 background were compared with age matched C57BL/6 and Sv129 wild type controls. The skin was treated twice a day for 6 days either with vehicle (acetone) alone or with GW1514 at 4 mM concentration in the vehicle. At the conclusion of the experiment, animals were sacrificed. Skin samples were taken and fixed with 4% formaldehyde in phosphate-buffered saline (PBS) and embedded in paraffin. Sections (6 μm) were collected on microscope slides and used for immunohistochemistry. Photographs were taken with a Zeiss microscope and digital images were captured and assembled by appropriate software. Fillagrin and loricrin protein expression were detected by immunohistochemistry using antibodies purchased from BabCo (Richmond, Calif.). The results are shown in FIG. 4.

Skin sections from GW1514 treated knockout (−/−) and wild type (+/+) mice both show increased immunohistochemical staining with filaggrin and loricrin antibodies. Thus, the experiments show that GW1514 stimulates keratinocyte differentiation in vivo, and this activation occurs completely independent of any PPAR-alpha activity.

### Example 4

This example shows that topical treatment with the PPAR-beta/delta activator GW1514 promotes the repair of a disrupted epidermal permeability barrier.

The stratum corneum is the outermost layer of the epidermis. It is the end product of keratinocyte differentiation and mediates epidermal barrier function. Since impaired barrier function is a feature of a number of common cutaneous disorders, the functional relevance of the pro-differentiating properties of GW1514 was tested by measuring restoration of permeability barrier homeostasis following perturbation in vivo.

The flanks of 6-8 week old hairless mice were treated either with vehicle alone (acetone) or with GW1514 at 4 mM concentration in vehicle twice daily for three days. Permeability of the epidermal barrier was then experimentally disrupted by the repeated application of cellophane tape, solvent extraction (repeated topical acetone), or by detergent treatment (repeated topical sodium dodecyl sulfate) until the transepidermal water loss (TEWL) reached 6-8 mg/cm²/h. Recovery of barrier function was determined by measurement of transepidermal water loss (TEWL) with an electrolytic water analyzer (MEECO®, Warrington, Pa.) at 3 and 6 hours after barrier disruption.

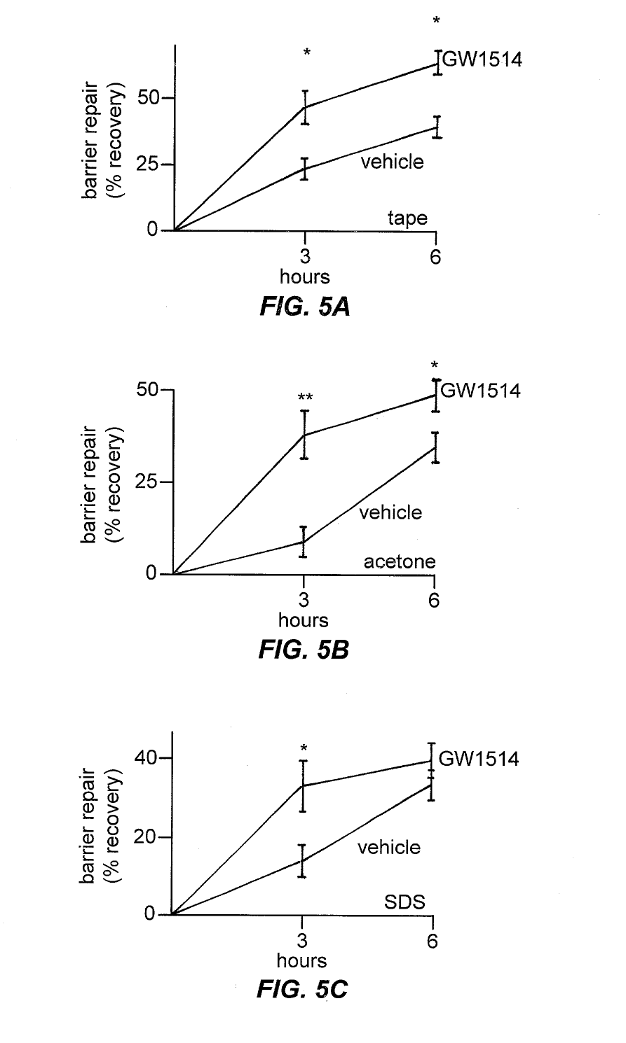

Barrier recovery is reported as percent barrier recovery from the initial induced defect. As can be seen in FIG. 5, regardless of the method of barrier perturbation, there was an accelerated recovery of permeability barrier function in skin pre-treated with GW1514. For example, at three hour post tape-strip, barrier function was almost 50% recovered in animals pretreated with topical applications of GW1514, whereas the untreated controls were less than 25% recovered at the same time point. Similarly, when barrier function was disrupted by repeated topical applications of acetone, three hours after the insult the GW1514 treated epidermis had recovered about 30% of its function, whereas untreated epidermis had only recovered about 10% of its barrier function.

Thus, topical application of the PPAR-beta/delta activator GW1514 to damaged epidermis has a beneficial effect on barrier repair kinetics.

### Example 5

This example shows that topical treatment of epidermis with the PPAR-beta/delta activator GW1514 following chronic barrier disruption and the induction of hyperplasia, results in epidermal differentiation without epidermal proliferation.

The skin of 6-8 week old hairless mice (Simonsen Laboratories, Gilroy, Calif.) was treated twice a day for 3 days with acetone soaked cotton balls until the transepidermal water loss reached 6-8 mg/cm²/h. This treatment resulted in epidermal hyperplasia (i.e. increased size due to increased cellular proliferation). After induction of hyperplasia, GW1514 at 4 mM concentration in acetone or acetone vehicle alone was applied topically for three days. At the completion of the experimental treatments, the animals were sacrificed and skin samples were taken and fixed with 4% formaldehyde in phosphate-buffered saline (PBS) and embedded in paraffin. Sections (6 μm) were collected on microscope slides and used for immunohistochemistry. Photographs were taken with a Zeiss microscope and digital images were captured and assembled by appropriate software. Fillagrin and loricrin protein expression were detected by immunohistochemistry using antibodies purchased from BabCo (Richmond, Calif.). Proliferating keratinocytes were quantified in sections by staining with biotinylated anti-proliferating cell nuclear antigen (PCNA) antibody (CalTag Laboratories, Burlingame, Calif.).

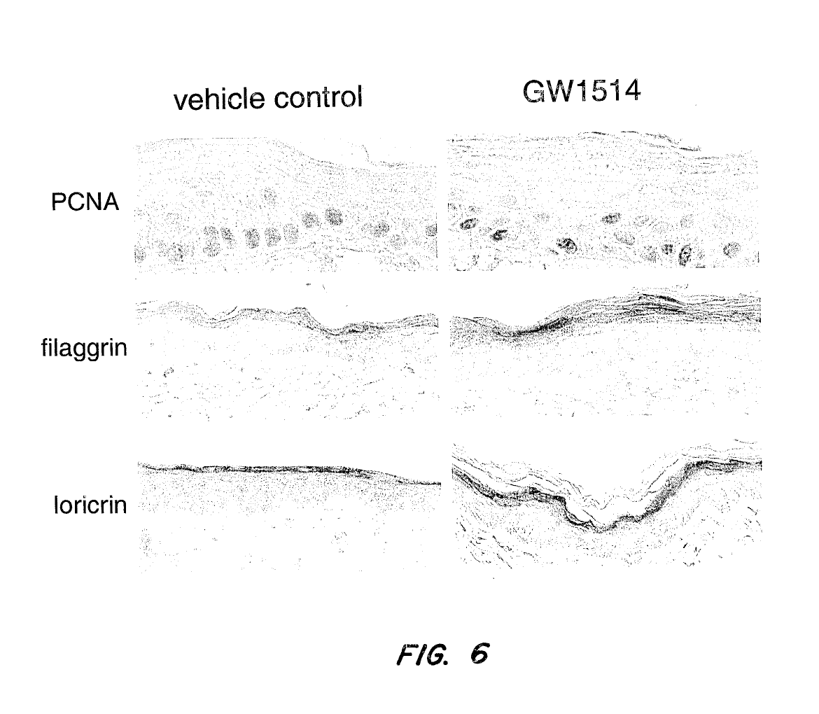

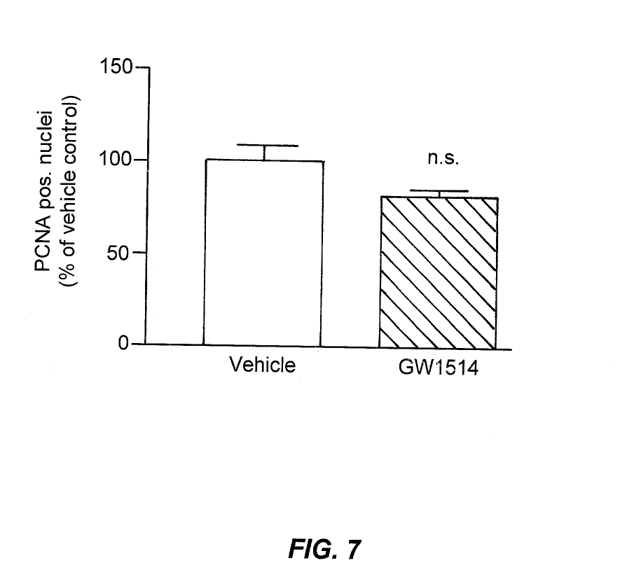

The results of the experiments are shown in FIGS. 6 and 7. As can be seen in the Figures, application of GW1514 to skin experimentally induced to hyperplasia resulted in an increase in the expression of the differentiation markers filaggrin and loricrin. However, application of GW1514 did not produce an increase in the number of proliferating nuclei as measured by PCNA staining.

Thus, these results suggest that PPAR-beta/delta activators can be important drugs for the treatment of diseases characterized by impaired differentiation and hyperproliferation (e.g. psoriasis) since they stimulate differentiation without also inducing proliferation. PPAR-beta/delta activators can also be useful in the treatment of conditions such as skin aging that involve impaired differentiation that is accompanied by normal or decreased proliferation.

### Example 6

This example demonstrates that, primary human keratinocytes cultured in the presence of substances known to stimulate inflammation, experience up-regulation of PPAR-beta/delta mRNA.

Second-passage keratinocytes isolated from newborn foreskins were cultured in serum-free keratinocyte growth medium (KGM; Clonetics, San Diego, Calif., USA). Cells were treated with the PPAR-beta/delta activator GW1514 (8 μM), tumor necrosis factor (TNF)-alpha (100 ng/ml), interleukin (IL)1-beta (50 ng/ml), lipopolysaccharide (LPS) (100 mg/ml) or UV-B irradiation (30 mJ/cm²) at pre-confluence. Control keratinocytes were treated with vehicle (0.05%) ethanol, or sham irradiated. UV exposure was performed in phosphate buffered saline which was replaced by serum free KGM after irradiation. For cytokine treatment, 0.1% bovine serum albumin was added to the medium.

PolyA RNA was purified using oligo(dT) cellulose (Amersham Pharmacia). RNA was electrophoresed through 1% agarose gels containing 2.2 M formaldehyde, and RNA integrity was visualized by ethidium bromide staining. The RNA was then transferred to a nylon membrane and blots were hybridized overnight at 65° C. with the appropriate ³²P-labeled cDNA probes. Probes were ³²P-labeled by random priming. Washes were performed in 0.1% SSC and 0.1 SDS for 20 minutes at room temperature and at 65° C., respectively. Autoradiography was performed at −70° C. Blots were probed with GAPDH to control for normal loading. Bands of the correct size were quantified by densitometry using Quantityone software (Bio-Rad Laboratories, Inc., Hercules, Calif., USA).

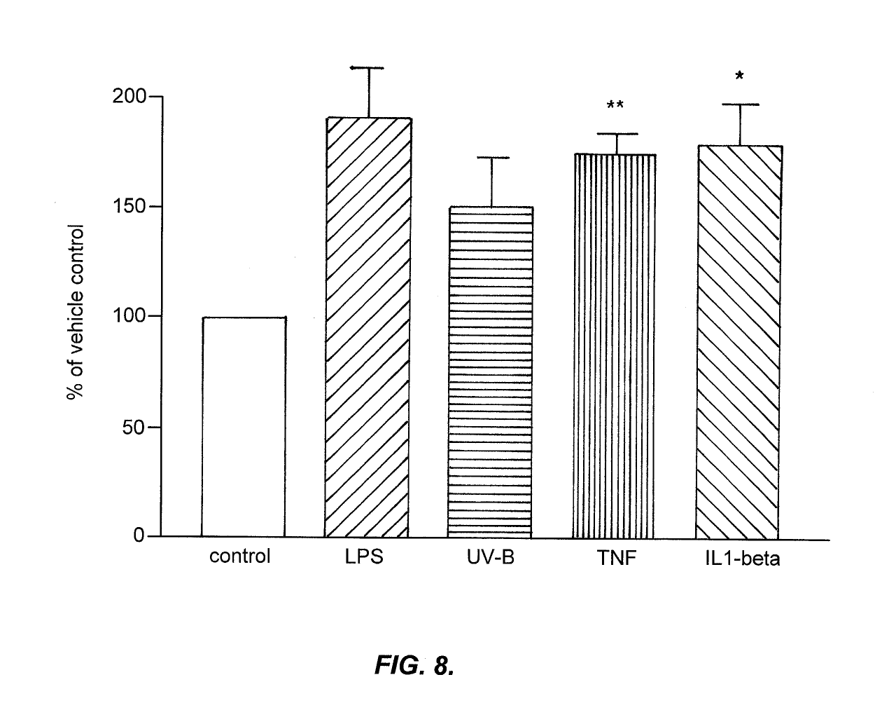

Results are shown in FIG. 8. Results of the northern analysis revealed an increase in PPAR-beta/delta mRNA levels under all the pro-inflammatory conditions tested. For example, levels of PPAR-beta/delta mRNA in lipopolysaccharide treated cells are nearly twice that of the untreated control. Similarly, PPAR-beta/delta mRNA levels in IL1-β treated cells are nearly twice that of the untreated control.

The up-regulation of PPAR-beta/delta mRNAin response to inflammatory stimuli suggests that increases in PPAR-beta/delta activity may be required to resolve the induced inflammation. Thus, these results suggest that PPAR-beta/delta is an ideal target for modulation of cutaneous inflammation in patients with inflammatory skin disorders.

### Example 7

This example demonstrates that topical application of the PPAR-beta/delta activator GW1514, is effective at reducing symptoms of inflammation that accompany experimentally induced contact dermatitis in an animal model.

In view of the selective increase in PPAR-beta/delta expression during inflammation (see e.g. Example 6 above) the effect of GW1514 in an animal model of inflammatory skin disease was investigated. Irritant contact dermatitis was experimentally induced by a single topical application of 10 μl of 0.03% (wt/vol in acetone)phorbol ester 12-O-tetradecanoylphorbol-13-acetate (TPA) to the ears of CD-1 mice. TPA was applied to the inner and outer surface of the left ears of male mice. The right ears were treated with vehicle alone (acetone). For topical treatment, GW1514 was dissolved in an acetone vehicle at a concentration of 4 mM. GW1514 was then applied to both the left and right ears at 45 minutes and 4 hours following inflammatory insult with 12-O-tetradecanoylphorbol-13-acetate (TPA). Ear thickness was measured with a digital caliper (Mitutoyo Corp., Tokyo, Japan).

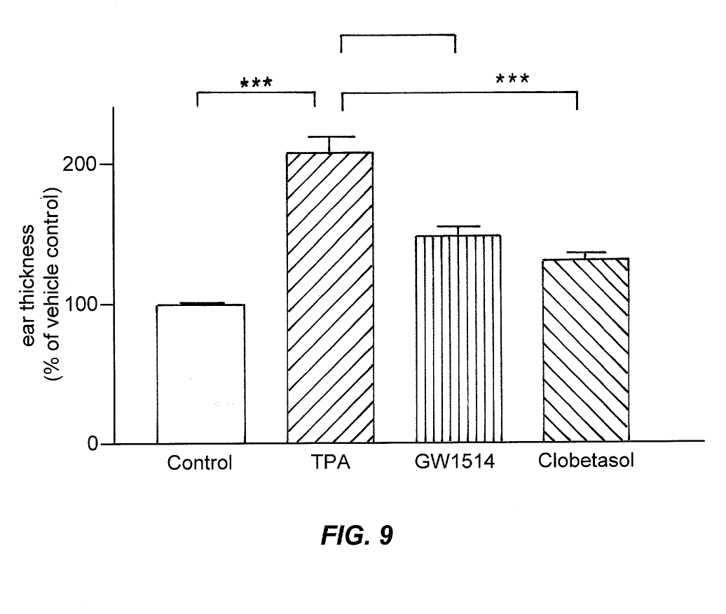

Both ear thickness (FIG. 9) and ear weight (not shown) increased approximately 2-fold following TPA treatment. The increase in ear thickness and weight was accompanied by a mixed cellular infiltrate containing neutrophils. As can be seen in the FIG. 9, treatment with GW1514 reduced the magnitude of the TPA induced increases in ear thickness, indicating an anti-inflammatory effect of GW1514. The reduction in inflammation produced by the PPAR-beta/delta agonist approached the anti-inflammatory effect of the topical glucocorticiod, clobetasol (FIG. 9).

Thus, topical application of the PPAR-betaldelta activator GW1514, provides an efficacious method for reducing the symptoms of inflammation associated contact dermatitis, and would be expected to be equally effective at reducing inflammation associated with other cutaneous disorders.

### Example 8

Psoriasis is a common skin disease characterized by epidermal hyperplasia, impaired differentiation, disturbed barrier function and inflammation. This example demonstrates that PPAR-beta/delta expression is up-regulated in psoriatic human skin when compared to normal human skin. Furthermore, this example demonstrates that while PPAR-beta/delta mRNA levels are increased in psoriasis, the levels of PPAR-alpha and PPAR-gamma mRNA are reduced.

Skin scrapings were obtained from psoriatic (n=9) and normal (n=8) epidermis of psoriasis patients. RNA was prepared from samples (50-100 mg tissue) utilizing triazol extraction procedure (Life Technologies, Gaithersburg, Md.). The expression of PPAR-beta/delta, PPAR-alpha and PPAR-gamma was then determined using quantitative PCR.

Quantitative PCR was carried out on one mg total RNA. cDNA was synthesized from the total RNA using SuperScript RT (Life Technologies). The cDNA was diluted in replicate plates, such that each well contained the cDNA from 50 ng of total RNA. SybrGreen PCR was conducted to measure the expression of PPAR-beta/delta or beta-actin genes using replicate 96-well plates. A 20 μL volume of SybrGreen PCR master mix (Applied Biosystems, Warrington, United Kingdom) containing 1 μl of 10 μM forward primer, 1 μl of 10 μM reverse primer, 5.5 μl water, was added to each well (Beckman Coulter, High Wycombe, United Kingdom). The PCR reaction was carried out on an ABI7700 Sequence Detector (Applied Biosystems) using the following PCR parameters: 50° C. for 2 minutes, 95° C. for 10 minutes and 40 cycles of 94° C. for 15 seconds, 60° C. for 1 minute. The level of mRNA-derived cDNA in each sample was estimated from the SybrGreen signal using genomic DNA calibration standards included in each experiment.

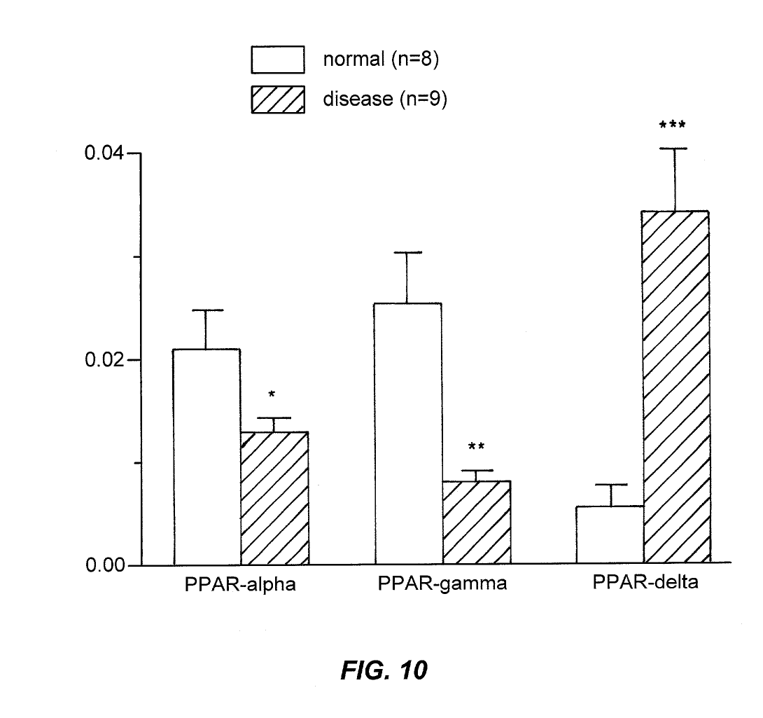

The results are shown in FIG. 10. The results of quantitative PCR analysis show that in lesional skin of psoriasis patients there is significant up-regulation of PPAR-beta/delta mRNA by comparison to normal skin. In contrast, PPAR-alpha and PPAR-gamma mRNA levels were down-regulated (FIG. 10). Thus, psoriatic lesions show selective subtype up-regulation of PPAR-beta/delta.

The results of these experiments, coupled with the findings presented in Example 6 showing increased levels of PPAR-beta/delta mRNA in response to inflammatory stimuli, and the results presented in Example 7 wherein inflammation is reduced by the application of PPAR-beta/delta activators, suggest that PPAR-beta/delta activators are effective treatments for diseases such as psoriasis, that involve cutaneous inflammation. Thus, the anti-inflammatory properties of GW1514 coupled with the increased expression of PPAR-beta/delta during inflammation suggest that compounds that activate PPAR-beta/delta will be promising treatment for inflammatory disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for treating the epidermis of a terrestrial mammalian subject suffering from a condition characterized by a perturbed epidermal barrier function, said method comprising applying to said epidermis a topical composition comprising a compound that is an activator of peroxisome proliferator-activated receptor δ but not of peroxisome proliferator-activated receptor α, in an amount effective in enhancing barrier development.

2. A method in accordance with claim 1 in which said compound is a member selected from the group consisting of (2-methyl-4-(((4-methyl-2-(4-trifluoromethylphenyl)-1,3-oxazol-5-yl)methyl)sulfanyl)phenoxy)acetic acid and (2-methyl-4-(((4-methyl-2-(4-trifluoromethylphenyl)-1,3-thiazol-5-yl)methyl)sulfanyl)phenoxy)acetic acid.

3. A method in accordance with claim 1 in which said compound is (2-methyl-4-(((4-methyl-2-(4-trifluoromethylphenyl)-1,3-oxazol-5-yl)methyl)sulfanyl)phenoxy)acetic acid.

4. A method in accordance with claim 1 in which said compound is (2-methyl-4-(((4-methyl-2-(4-trifluoromethylphenyl)-1,3-thiazol-5-yl)methyl)sulfanyl)phenoxy)acetic acid.

5. A method in accordance with claim 1 in which the concentration of said compound in said topical composition is from about 1 mM to about 50 mM.

6. A method in accordance with claim 1 in which the concentration of said compound in said topical composition is from about 1 mM to about 20 mM.

7. A method for treating the epidermis or mucous membrane of a terrestrial mammalian subject suffering from a condition of disturbed differentiation or excess proliferation, said method comprising topically administering to said epidermis or mucous membrane a topical composition comprising a compound that is an activator of peroxisome proliferator-activated receptor δ but not of peroxisome proliferator-activated receptor α, in an amount effective in normalizing said condition.

8. A method in accordance with claim 7 in which said compound is a member selected from the group consisting of (2-methyl-4-(((4-methyl-2-(4-trifluoromethylphenyl)-1,3-oxazol-5-yl)methyl)sulfanyl)phenoxy)acetic acid and (2-methyl-4-(((4-methyl-2-(4-trifluoromethylphenyl)-1,3-thiazol-5-yl)methyl)sulfanyl)phenoxy)acetic acid.

9. A method in accordance with claim 7 in which said compound is (2-methyl-4-(((4-methyl-2-(4-trifluoromethylphenyl)-1,3-oxazol-5-yl)methyl)sulfanyl)phenoxy)acetic acid.

10. A method in accordance with claim 7 in which said compound is (2-methyl-4-(((4-methyl-2-(4-trifluoromethylphenyl)-1,3-thiazol-5-yl)methyl)sulfanyl)phenoxy)acetic acid.

11. A method in accordance with claim 7 in which the concentration of said compound in said topical composition is from about 1 mM to about 50 mM.

12. A method in accordance with claim 7 in which the concentration of said compound in said topical composition is from about 1 mM to about 20 mM.

13. A method for treating the epidermis or mucous membrane of a terrestrial mammalian subject suffering from a condition of epidermal inflammation, said method comprising topically administering to said epidermis or mucous membrane a topical composition comprising a compound that is an activator of peroxisome proliferator-activated receptor δ but not of peroxisome proliferator-activated receptor α, in an amount effective in normalizing said condition.

14. A method in accordance with claim 13 in which said compound is a member selected from the group consisting of (2-methyl-4-(((4-methyl-2-(4-trifluoromethylphenyl)-1,3-oxazol-5-yl)methyl)sulfanyl)phenoxy)acetic acid and (2-methyl-4-(((4-methyl-2-(4-trifluoromethylphenyl)-1,3-thiazol-5-yl)methyl)sulfanyl)phenoxy)acetic acid.

15. A method in accordance with claim 13 in which said compound is (2-methyl-4-(((4-methyl-2-(4-trifluoromethylphenyl)-1,3-oxazol-5-yl)methyl)sulfanyl)phenoxy)acetic acid.

16. A method in accordance with claim 13 in which said compound is (2-methyl-4-(((4-methyl-2-(4-trifluoromethylphenyl)-1,3-thiazol-5-yl)methyl)sulfanyl)phenoxy)acetic acid.

17. A method in accordance with claim 13 in which the concentration of said compound in said topical composition is from about 1 mM to about 50 mM.

18. A method in accordance with claim 13 in which the concentration of said compound in said topical composition is from about 1 mM to about 20 mM.
